(19) United States
(12) Patent Application Publication
(10) Pub. No.: US 2025/0134451 A1
(43) Pub. Date: May 1, 2025

(54) BIOFEEDBACK COGNITIVE BEHAVIORAL THERAPY FOR INSOMNIA

(71) Applicant: ResMed Digital Health Inc., San Diego, CA (US)

(72) Inventors: Redmond Shouldice, Dublin (IE); Kieran Conway, Dublin (IE); Michael Wren, Dublin (IE); Stephen Dodd, Dublin (IE)

(21) Appl. No.: 18/687,788

(22) PCT Filed: Aug. 24, 2022
(86) PCT No.: PCT/IB2022/057940

§ 371 Date: Feb. 28, 2024

Related U.S. Application Data
(60) Provisional application No. 63/238,437, filed on Aug. 30, 2021.

(51) Int. Cl. A61B 5/00; A61B 5/11; G16H 10/60; G16H 20/70

(52) U.S. Cl. CPC A61B 5/4806; A61B 5/11; G16H 10/60; G16H 20/70

(57) ABSTRACT

Intelligent systems and methods for facilitating insomnia therapy are disclosed. Sensor data (e.g., non-contact sensor data) can be used to determine physiological parameter(s), such as sleep-related physiological parameter(s), which can be used to generate a sleep disorder prediction. The sleep disorder prediction can be used, along with an identified sleep therapy plan, to generate and facilitate application of (e.g., present to a user or automatically apply) a sleep therapy recommendation. When sleep apnea is predicted in concert with an identified cognitive behavioral therapy for insomnia (CBTi) plan, a warning can be presented to the user to not engage in certain CBTi therapies. Sensor data can also be used to automatically update therapy parameter(s) of an ongoing sleep therapy plan, such as in realtime.

### CROSS-REFERENCE TO RELATED APPLICATIONS

This application claims the benefit of, and priority to, U.S. Provisional Patent Application No. 63/238,437 filed on Aug. 30, 2021, which is hereby incorporated by reference herein in its entirety.

### TECHNICAL FIELD

The present disclosure relates generally to systems and methods for improving insomnia therapy, and more particularly, to systems and methods for providing intelligent insomnia therapy and prescreening.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), shift work sleep disorder, non-24-hour sleep-wake disorder, hypertension, diabetes, stroke, insomnia, and chest wall disorders.

In some cases, individuals may suffer from multiple disorders and may seek treatment for one or more disorders. For some disorders, treatment can include use of a respiratory therapy system. Some individuals may undergo a sleep therapy plan to improve one or more of the disorders. Often, sleep therapy plans involve repeated sessions with a healthcare professional and completion of long questionnaires to evaluate the success of a sleep therapy plan and determine what updates may need to be made to a sleep therapy plan.

One example of sleep therapy that is helpful for treating insomnia is cognitive behavior therapy for insomnia (CBTi). CBTi can involve a multi-part sleep therapy plan that can be implemented in various ways. CBTi often involves manually preparing extensive logs (which are subject to intentional or non-intentional inaccuracies) and working with a healthcare professional to make periodic adjustments to how the individual approaches sleep. For example, a common CBTi technique is to undergo sleep restriction, in which the individual purposefully limits sleep sessions to a short window of time by going to sleep and awakening at certain times. Over time, the individual may be able to achieve better sleep during that short window. Afterwards, as the user increases the window to a longer timeframe, the user is ideally able to achieve that same higher-quality sleep for longer durations. While sleep restriction can be beneficial for some individuals, it can be dangerous for those with certain other diagnosed or undiagnosed sleep disorders, such as those with comorbid insomnia and OSA (COMISA). For such an individual, whether diagnosed with COMISA or not, engaging in sleep restriction may not have the expected outcome, and may in fact result in harmful side effects. The OSA can cause the shorter amount of sleep obtained by the individual to be interrupted (e.g., by apneas) and otherwise by less restful than expected. As a result, the individual may be especially fatigued and/or accident prone the following day, which can prove dangerous or even deadly to the individual. If pharmacological input is also part of CBTi, undiagnosed SDB, such as OSA, can be worsened if a specific medication suppresses breathing drive. The present disclosure is directed to solving these and other problems.

### SUMMARY

According to some implementations of the present disclosure, a method includes receiving sensor data from one or more sensors. The sensor data is associated with a user engaging in a sleep therapy plan, such as CBTi. The method further includes receiving one or more therapy parameters associated with the sleep therapy plan. The method further includes dynamically generating at least one updated therapy parameter associated with the sleep therapy plan based at least in part on the one or more therapy parameters and the received sensor data. The method further includes presenting the at least one updated therapy parameter to affect the sleep therapy plan.

According to some implementations of the present disclosure, a method includes receiving sensor data from one or more sensors. The sensor data is associated with a user. The method further includes determining one or more physiological parameters based at least in part on the received sensor data. The method further includes generating a sleep disorder prediction based at least in part on the one or more physiological parameters. The method further includes identifying a future sleep therapy plan associated with the user. The method further includes generating a sleep therapy plan recommendation based at least in part on the generated sleep disorder prediction and the identified sleep therapy plan. The method further includes facilitating application of the sleep therapy plan recommendation to the future sleep therapy plan prior to implementation of the future sleep therapy plan.

According to some implementations of the present disclosure, a system includes an electronic interface, a memory, and a control system. The electronic interface is configured to receive sensor data associated with a user engaging in a sleep therapy plan. The memory stores machine-readable instructions. The control system includes one or more processors configured to execute the machine-readable instructions to receive one or more therapy parameters associated with the sleep therapy plan. The control system is further configured to dynamically generate at least one updated therapy parameter associated with the sleep therapy plan based at least in part on the one or more therapy parameters and the received sensor data. The control system is further configured to apply the at least one updated therapy parameter to affect the sleep therapy plan.

According to some implementations of the present disclosure, a system includes an electronic interface, a memory, and a control system. The electronic interface is configured to receive sensor data associated with a user. The memory stores machine-readable instructions. The control system includes one or more processors configured to execute the machine-readable instructions to determine one or more physiological parameters based at least in part on the received sensor data. The control system is further configured to generate a sleep disorder prediction based at least in part on the one or more physiological parameters. The control system is further configured to identify a future sleep therapy plan associated with the user. The control system is further configured to generate a sleep therapy plan recommendation based at least in part on the generated sleep disorder prediction and the identified sleep therapy plan. The control system is further configured to facilitate application of the sleep therapy plan recommendation to the future sleep therapy plan prior to implementation of the future sleep therapy plan.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 is a flowchart depicting a process for updating a sleep therapy plan according to some implementations of the present disclosure.

FIG. 6 is a timeline diagram depicting dynamic updating of a sleep therapy plan during a sleep session, according to some implementations of the present disclosure.

FIG. 7 is a flowchart depicting a process for generating a sleep therapy plan recommendation according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

As disclosed in further detail herein, intelligent systems and methods for facilitating insomnia therapy are disclosed, such as to pre-screen for a future sleep therapy plan, help establish a future sleep therapy plan, or automatically update an existing sleep therapy plan. Sensor data (e.g., non-contact sensor data) can be used to determine physiological parameter(s), such as sleep-related physiological parameter(s), which can be used to generate a sleep disorder prediction. The sleep disorder prediction can be used, along with an identified sleep therapy plan, to generate and facilitate application of (e.g., present to a user or automatically apply) a sleep therapy recommendation. When sleep apnea is predicted in concert with an identified cognitive behavioral therapy for insomnia (CBTi) plan, a warning can be presented to the user to not engage in certain CBTi therapies. Sensor data can also be used to automatically update therapy parameter(s) of an ongoing sleep therapy plan, optionally in realtime or near realtime.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), shift work sleep disorder, non-24-hour sleep-wake disorder, hypertension, diabetes, stroke, insomnia, parainsomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Rapid eye movement behavior disorder (RBD) is characterized by a lack of muscle atonia during REM sleep, and in more severe cases, movement and speech produced by an individual during REM sleep stages. RBD can sometimes be accompanied by dream enactment behavior (DEB), where the individual acts out dreams they may be having, sometimes resulting in injuries to themselves or their partners. RBD is often a precursor to a subclass of neuro-degenerative disorders, such as Parkinson's disease, Lewis Body Dementia, and Multiple System Atrophy. Typically, RBD is diagnosed in a sleep laboratory via polysomnography. This process can be expensive, and often occurs late in the evolution process of the disease, when mitigating therapies are difficult to adopt and/or less effective. Monitoring an individual during sleep in a home environment or other common sleeping environment can beneficially be used to identify whether the individual is suffering from RBD or DEB.

Shift work sleep disorder is a circadian rhythm sleep disorder characterized by a circadian misalignment related to a work schedule that overlaps with a traditional sleep-wake cycle. This disorder often presents as insomnia when attempting to sleep and/or excessive sleepiness while working for an individual engaging in shift work. Shift work can involve working nights (e.g., after 7 pm), working early mornings (e.g., before 6 am), and working rotating shifts. Left untreated, shift work sleep disorder can result in complications ranging from light to serious, including mood problems, poor work performance, higher risk of accident, and others.

Non-24-hour sleep-wake disorder (N24SWD), formally known as free-running rhythm disorder or hypernychthemeral syndrome, is a circadian rhythm sleep disorder in which the body clock becomes desynchronized from the environment. An individual suffering from N24SWD will have a circadian rhythm that is shorter or longer than 24 hours, which causes sleep and wake times to be pushed progressively earlier or later. Over time, the circadian rhythm can become desynchronized from regular daylight hours, which can cause problematic fluctuations in mood, appetite, and alertness. Left untreated, N24SWD can result in further health consequences and other complications.

Many individuals suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep. Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower mental (and/or physical) performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity.

Co-morbid Insomnia and Sleep Apnea (COMISA) refers to a type of insomnia where the subject experiences both insomnia and obstructive sleep apnea (OSA). OSA can be measured based on an Apnea-Hypopnea Index (AHI) and/or oxygen desaturation levels. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild OSA. An AHI that is greater than or equal to 15, but less than 30 is considered indicative of moderate OSA. An AHI that is greater than or equal to 30 is considered indicative of severe OSA. In children, an AHI that is greater than 1 is considered abnormal.

Insomnia can also be categorized based on its duration. For example, insomnia symptoms are considered acute or transient if they occur for less than 3 months. Conversely, insomnia symptoms are considered chronic or persistent if they occur for 3 months or more, for example. Persistent/chronic insomnia symptoms often require a different treatment path than acute/transient insomnia symptoms.

Known risk factors for insomnia include gender (e.g., insomnia is more common in females than males), family history, and stress exposure (e.g., severe and chronic life events). Age is a potential risk factor for insomnia. For example, sleep-onset insomnia is more common in young adults, while sleep-maintenance insomnia is more common in middle-aged and older adults. Other potential risk factors for insomnia include race, geography (e.g., living in geographic areas with longer winters), altitude, and/or other sociodemographic factors (e.g. socioeconomic status, employment, educational attainment, self-rated health, etc.).

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

Conventionally, diagnosing or screening insomnia (including identifying a type or insomnia and/or specific symptoms) involves a series of steps. Often, the screening process begins with a subjective complaint from a patient (e.g., they cannot fall or stay sleep).

Next, the clinician evaluates the subjective complaint using a checklist including insomnia symptoms, factors that influence insomnia symptoms, health factors, and social factors. Insomnia symptoms can include, for example, age of onset, precipitating event(s), onset time, current symptoms (e.g., sleep-onset, sleep-maintenance, late insomnia), frequency of symptoms (e.g., every night, episodic, specific nights, situation specific, or seasonal variation), course since onset of symptoms (e.g., change in severity and/or relative emergence of symptoms), and/or perceived daytime consequences. Factors that influence insomnia symptoms include, for example, past and current treatments (including their efficacy), factors that improve or ameliorate symptoms, factors that exacerbate insomnia (e.g., stress or schedule changes), factors that maintain insomnia including behavioral factors (e.g., going to bed too early, getting extra sleep on weekends, drinking alcohol, etc.) and cognitive factors (e.g., unhelpful beliefs about sleep, worry about consequences of insomnia, fear of poor sleep, etc.). Health factors include medical disorders and symptoms, conditions that interfere with sleep (e.g., pain, discomfort, treatments), and pharmacological considerations (e.g., alerting and sedating effects of medications). Social factors include work schedules that are incompatible with sleep, arriving home late without time to wind down, family and social responsibilities at night (e.g., taking care of children or elderly), stressful life events (e.g., past stressful events may be precipitants and current stressful events may be perpetuators), and/or sleeping with pets.

After the clinician completes the checklist and evaluates the insomnia symptoms, factors that influence the symptoms, health factors, and/or social factors, the patient is often directed to create a daily sleep diary and/or fill out a questionnaire (e.g., Insomnia Severity Index or Pittsburgh Sleep Quality Index). Thus, this conventional approach to insomnia screening and diagnosis is susceptible to error(s) because it relies on subjective complaints rather than objective sleep assessment. There may be a disconnect between patient's subjective complaint(s) and the actual sleep due to sleep state misperception (paradoxical insomnia).

In addition, the conventional approach to insomnia diagnosis does not rule out other sleep-related disorders such as, for example, Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. While these other sleep-related disorders may have similar symptoms as insomnia, distinguishing these other sleep-related disorders from insomnia is useful for tailoring an effective treatment plan distinguishing characteristics that may call for different treatments. For example, fatigue is generally a feature of insomnia, whereas excessive daytime sleepiness is a characteristic feature of other disorders (e.g., PLMD) and reflects a physiological propensity to fall asleep unintentionally.

Once diagnosed, insomnia can be managed or treated using a variety of techniques or providing recommendations to the patient. A plan of therapy used to treat insomnia, or other sleep-related disorders, can be known as a sleep therapy plan. For insomnia, the patient might be encouraged or recommended to generally practice healthy sleep habits (e.g., plenty of exercise and daytime activity, have a routine, no bed during the day, eat dinner early, relax before bedtime, avoid caffeine in the afternoon, avoid alcohol, make bedroom comfortable, remove bedroom distractions, get out of bed if not sleepy, try to wake up at the same time each day regardless of bed time) or discouraged from certain habits (e.g., do not work in bed, do not go to bed too early, do not go to bed if not tired). The patient can additionally or alternatively be treated using sleep medicine and medical therapy such as prescription sleep aids, over-the-counter sleep aids, and/or at-home herbal remedies.

The patient can also be treated using cognitive behavior therapy (CBT) or cognitive behavior therapy for insomnia (CBT-I), which is a type of sleep therapy plan that generally includes sleep hygiene education, relaxation therapy, stimulus control, sleep restriction, and sleep management tools and devices. Sleep restriction is a method designed to limit time in bed (the sleep window or duration) to actual sleep, strengthening the homeostatic sleep drive. The sleep window can be gradually increased over a period of days or weeks until the patient achieves an optimal sleep duration. Stimulus control includes providing the patient a set of instructions designed to reinforce the association between the bed and bedroom with sleep and to reestablish a consistent sleep-wake schedule (e.g., go to bed only when sleepy, get out of bed when unable to sleep, use the bed for sleep only (e.g., no reading or watching TV), wake up at the same time each morning, no napping, etc.) Relaxation training includes clinical procedures aimed at reducing autonomic arousal, muscle tension, and intrusive thoughts that interfere with sleep (e.g., using progressive muscle relaxation). Cognitive therapy is a psychological approach designed to reduce excessive worrying about sleep and reframe unhelpful beliefs about insomnia and its daytime consequences (e.g., using Socratic question, behavioral experiences, and paradoxical intention techniques). Sleep hygiene education includes general guidelines about health practices (e.g., diet, exercise, substance use) and environmental factors (e.g., light, noise, excessive temperature) that may interfere with sleep. Mindfulness-based interventions can include, for example, meditation.

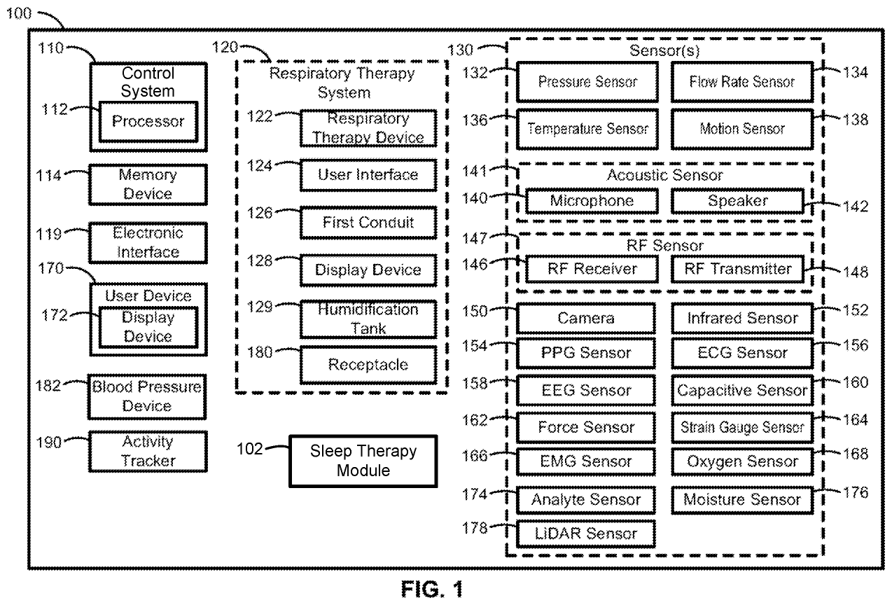

Referring to FIG. 1, a functional block diagram is illustrated, of a system 100 for facilitating a sleep therapy plan for a user, such as a user of a respiratory therapy system. The system 100 includes a sleep therapy module 102, a control system 110, a memory device 114, an electronic interface 119, one or more sensors 130, and one or more user devices 170. In some implementations, the system 100 further optionally includes a respiratory therapy system 120, a blood pressure device 182, an activity tracker 190, or any combination thereof.

The sleep therapy module 102 receives, generates, and/or updates information pertaining to a sleep therapy plan, such as therapy parameters of a sleep therapy plan, as disclosed in further detail herein. Some or all of the sleep therapy module 102 can be implemented by and/or make use of any other elements of system 100. In some cases, sleep therapy module 102 can communicate with one or more user devices 170 to present information (e.g., a sleep therapy plan recommendation or an updated therapy parameter) and/or automatically apply updates (e.g., automatically update a therapy parameter and/or otherwise automatically adjust a sleep therapy plan). In some cases, sleep therapy module 102 can be integrated into a user device 170, such as a general purpose user device (e.g., a smartphone) or a specific purpose user device (e.g., a user device designed and/or sold for implementing a sleep therapy plan).

The control system 110 includes one or more processors 112 (hereinafter, processor 112). The control system 110 is generally used to control (e.g., actuate) the various components of the system 100 and/or analyze data obtained and/or generated by the components of the system 100 (e.g., sleep therapy module 102). The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, the activity tracker 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, the activity tracker 190, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more sleep sessions), sleep therapy plan information (e.g., therapy parameters) associated with the user, or any combination thereof. The demographic information can include, for example, information indicative of an age of the user, a gender of the user, a race of the user, an ethnicity of the user, a geographic location of the user, a travel history of the user, a relationship status, a status of whether the user has one or more pets, a status of whether the user has a family, a family history of health conditions, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective therapy score (e.g., poor, average, excellent), a self-reported subjective stress level of the user, a self-reported subjective fatigue level of the user, a self-reported subjective health status of the user, a recent life event experienced by the user, or any combination thereof. The sleep therapy plan information can include various information associated with one or more sleep therapy plans, such as information regarding the user's historical sleep therapy plans, the effects of one or more historical sleep sessions using such sleep therapy plans, customized therapy parameters (e.g., sleep therapy plan preferences or other parameters) associated with the user, and the like. The user profile information can be updated at any time, such as daily (e.g. between sleep sessions), weekly, monthly or yearly. In some implementations, the memory device 114 stores media content that can be displayed on the display device 128 and/or the display device 172.

The electronic interface 119 is configured to receive data (e.g., physiological data, environmental data, pharmacological data, flow rate data, pressure data, motion data, acoustic data, etc.) from the one or more sensors 130 such that the data can be stored in the memory device 114 and/or analyzed by the processor 112 of the control system 110. The received data, such as physiological data, flow rate data, pressure data, motion data, acoustic data, etc., may be used to determine and/or calculate one or more parameters associated with the user, the user's environment, or the like. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The respiratory therapy system 120 can include a respiratory pressure therapy (RPT) device 122 (referred to herein as respiratory device 122), a user interface 124, a conduit 126 (also referred to as a tube or an air circuit), a display device 128, a humidification tank 129, a receptacle 180 or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver pressurized air at a pressure of at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory device 122, the user interface 124, and the conduit 126 form an air pathway fluidly coupled with an airway of the user. The pressurized air also increases the user's oxygen intake during sleep.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

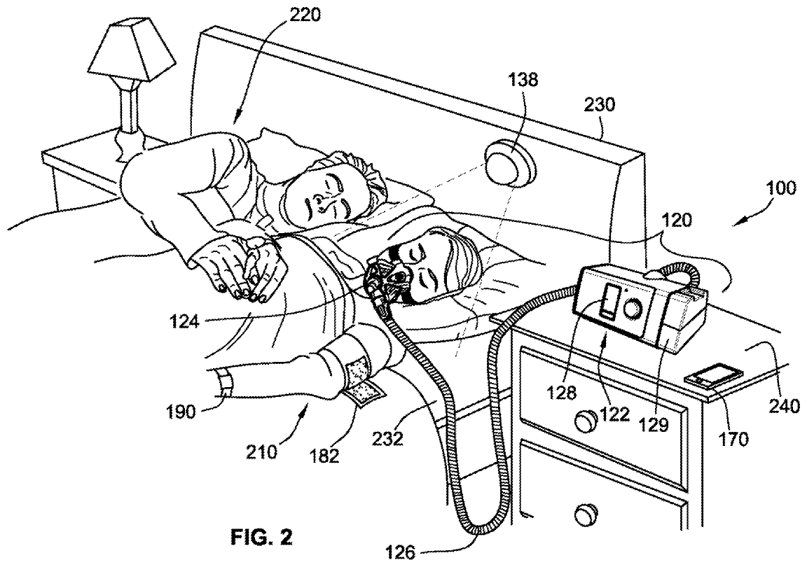

As shown in FIG. 2, in some implementations, the user interface 124 is or includes a facial mask (e.g., a full face mask) that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of the respiratory therapy system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, a humidity sensor, a temperature sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score and/or a therapy score (such as a my Air™ score, such as described in WO 2016/061629, which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122. The humidification tank 129 includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory device 122 or the conduit 126 can include a waterless humidifier. The waterless humidifier can incorporate sensors that interface with other sensor positioned elsewhere in system 100.

In some implementations, the system 100 can be used to deliver at least a portion of a substance from a receptacle 180 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) any combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. A motion sensor 138, a blood pressure device 182, and an activity tracker 190 are shown, although any one or more sensors 130 can be used to generate or monitor various parameters during a respiratory therapy, sleep therapy, sleeping, and/or resting session of the user 210. Certain aspects of the present disclosure can relate to facilitating sleep therapy for any individual, such as an individual using a respiratory therapy device (e.g., user 210) or an individual not using a respiratory therapy device (e.g., bed partner 220).

The user interface 124 is a facial mask (e.g., a full face mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps (e.g., including hook and loop fasteners) for positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user 210.

The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, their airway can narrow or collapse during sleep, reducing oxygen intake, and forcing them to wake up and/or otherwise disrupt their sleep. The CPAP machine prevents the airway from narrowing or collapsing, thus minimizing the occurrences where she wakes up or is otherwise disturbed due to reduction in oxygen intake. While the respiratory device 122 strives to maintain a medically prescribed air pressure or pressures during sleep, the user can experience sleep discomfort due to the therapy.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a Light Detection and Ranging (LiDAR) sensor 178, an electrodermal sensor, an accelerometer, an electrooculography (EOG) sensor, a light sensor, a humidity sensor, an air quality sensor, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the Light Detection and Ranging (LiDAR) sensor 178 more generally, the one or more sensors 130 can include any combination and any number of each of the sensors described and/or shown herein.

Data from room environment sensors can also be used, such as to extract environmental parameters from sensor data. Example environmental parameters can include temperature before and/or throughout a sleep session (e.g., too warm, too cold), humidity (e.g., too high, too low), pollution levels (e.g., an amount and/or concentration of CO₂ and/or particulates being under or over a threshold), light levels (e.g., too bright, not using blackout blinds, too much blue light before falling asleep), and sound levels (e.g., above a threshold, types of sources, linked to interruptions in sleep, snoring of a partner). These parameters can be obtained via sensors on a respiratory therapy device, via sensors on a smartphone (e.g., connected via Bluetooth or internet), or via separate sensors (such as connected to a home automation system). An air quality sensor can also detect other types of pollution in the room that cause allergies, such as from pets, dust mites, and so forth-and where the room could benefit from air filtration in order to facilitate engagement of a sleep therapy plan.

Health record data (e.g., physical and/or mental) can also be used in the facilitation of engaging in a sleep therapy plan. For example, information about one or more medical conditions, including diagnosis information and/or treatment information, can be used when determining how to modify a therapy parameter of a sleep therapy plan or when determining whether or not a sleep therapy plan is suitable or recommended for the user. Variation in a user's response to a sleep therapy plan and/or changes to a sleep therapy plan can also relate to health (such as a change due to the onset or offset of illness, such as a respiratory issue, and/or due to a change in an underlying condition such as a co-morbid chronic condition).

In some cases, one or more sensors 130 can be used to obtain pharmacological data (e.g., pharmacological parameters), such as information about whether or not a user has taken medication, what medication was taken by the user, how much medication the user took, the timing of when the user took the medication, and the like. In some cases, pharmacological data can be extracted from one or more sensors associated with the user or associated with a pharmacological container. In some cases, a pharmacological container sensor can be used, in which case the pharmacological container may include a sensor incorporated therein or otherwise associated therewith (e.g., a weight sensor, such as force sensor 162, coupled to the pharmacological container to identify when the user accesses the pharmacological container). In a further example, a camera (e.g., camera 150) can use machine vision to identify a pattern of actions associated with a user taking certain medication.

An analysis of sleep quality based on processing of sensors can be used, such as to check for insomnia (including due to hyper-arousal, as checked via a person's temperature and/or heart rate elevation). The system can match detected possible discomfort factors to acute insomnia, such as the onset of insomnia due to a difficulty in falling asleep, staying asleep, or waking up earlier than expected or desired. Sleep quality can include information associated with sleep efficiency as well as other quality-related factors (e.g., time spent in certain sleep stages, total sleep time, and the like).

As described herein, the system 100 generally can be used to generate data (e.g., physiological data, environmental data, pharmacological data, flow rate data, pressure data, motion data, acoustic data, etc.) associated with a user (e.g., a user of the respiratory therapy system 120 shown in FIG. 2 or any other suitable user) before, during, and/or after a sleep session. The generated data can be analyzed to extract one or more parameters, including physiological parameters (e.g., heart rate, heart rate variability, temperature, temperature variability, respiration rate, respiration rate variability, breath morphology, EEG activity, EMG activity, ECG data, and the like), environmental parameters associated with the user's environment (e.g., a sleep environment), pharmacological parameters (e.g., parameters associated with the user's taking of medication), and the like. Physiological parameters can include sleep-related parameters associated with a sleep session as well as non-sleep related parameters. Examples of one or more sleep-related parameters that can be determined for a user during the sleep session include an Apnea-Hypopnea Index (AHI) score, a sleep score, a therapy score, a flow signal, a pressure signal, a respiration signal, a respiration rate, an inspiration amplitude, an expiration amplitude, an inspiration-expiration ratio, a number of events (e.g. apnea events) per hour, a pattern of events, a sleep state and/or sleep stage, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, environmental data, pharmacological data, flow rate data, pressure data, motion data, acoustic data, etc. In some implementations, the data generated by one or more of the sensors 130 can be used by the control system 110 to determine the duration of sleep and sleep quality of user 210. For example, a sleep-wake signal associated with the user 210 during the sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory device 122, or any combination thereof during the sleep session.

The event(s) can include snoring, apneas (e.g., central apneas, obstructive apneas, mixed apneas, and hypopneas), a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. In some implementations, mouth leak can include continuous mouth leak, or valve-like mouth leak (i.e. varying over the breath duration) where the lips of a user, typically using a nasal/nasal pillows mask, pop open on expiration. Mouth leak can lead to dryness of the mouth, bad breath, and is sometimes colloquially referred to as “sandpaper mouth.”

The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

The data generated by the one or more sensors 130 (e.g., physiological data, environmental data, pharmacological data, flow rate data, pressure data, motion data, acoustic data, etc.) can also be used to determine a respiration signal. The respiration signal is generally indicative of respiration or breathing of the user. The respiration signal can be indicative of, for example, a respiration rate, a respiration rate variability, an inspiration amplitude, an expiration amplitude, an inspiration-expiration ratio, and other respiration-related parameters, as well as any combination thereof. In some cases, during a sleep session, the respiration signal can include a number of events per hour (e.g., during sleep), a pattern of events, pressure settings of the respiratory device 122, or any combination thereof. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, labored breathing, an asthma attack, an epileptic episode, a seizure, or any combination thereof.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory device 122. the user interface 124, or the conduit 126. The pressure sensor 132 can be used to determine an air pressure in the respiratory device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The flow rate sensor 134 can be used to generate flow rate data associated with the user 210 (FIG. 2) of the respiratory device 122 during the sleep session. Examples of flow rate sensors (such as, for example, the flow rate sensor 134) are described in WO 2012/012835, which is hereby incorporated by reference herein in its entirety. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state or sleep stage of the user; for example, via a respiratory movement of the user. In some implementations, the motion data from the motion sensor 138 can be used in conjunction with additional data from another sensor 130 to determine the sleep state or sleep stage of the user. In some implementations, the motion data can be used to determine a location, a body position, and/or a change in body position of the user.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep related parameters, which may include one or more events (e.g., respiratory events), as described in further detail herein. The microphone 140 can be coupled to or integrated in the respiratory device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 (e.g., in response to an identified body position and/or a change in body position). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g. a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (including e.g., an identified body position and/or a change in body position) and/or respiration-related parameters described in herein such as, for example, a respiration signal (from which e.g., breath morphology may be determined), a respiration rate, an inspiration amplitude, an expiration amplitude, an inspiration-expiration ratio, a number of events per hour, a pattern of events, a sleep state, a sleep stage, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session.

The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near the user 210's mouth to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the user 210's nose to detect analytes in breath exhaled through the user's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the user 210's mouth or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be positioned in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, an orientation sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not physically and/or communicatively coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more parameters, such as physiological parameters, environmental parameters, pharmacological parameters, and the like, as disclosed in further detail herein. In some cases, one or more physiological parameters can include a respiration signal, a respiration rate, a respiration pattern or morphology, respiration rate variability, an inspiration amplitude, an expiration amplitude, an inspiration-expiration ratio, a length of time between breaths, a time of maximal inspiration, a time of maximal expiration, a forced breath parameter (e.g., distinguishing releasing breath from forced exhalation), an occurrence of one or more events, a number of events per hour, a pattern of events, a sleep state, a sleep stage, an apnea-hypopnea index (AHI), a heart rate, heart rate variability, movement of the user 210, temperature, EEG activity, EMG activity, ECG data, a sympathetic response parameter, a parasympathetic response parameter or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these physiological parameters are sleep-related parameters, although in some cases the data from the one or more sensors 130 can be analyzed to determine one or more non-physiological parameters, such as non-physiological sleep-related parameters. Non-physiological parameters can include environmental parameters and pharmacological parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s), optionally with a display, such as Google Home™, Google Nest™, Amazon Echo™, Amazon Echo Show™, Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating physiological data for determining one or more blood pressure measurements associated with a user. The blood pressure device 182 can include at least one of the one or more sensors 130 to measure, for example, a systolic blood pressure component and/or a diastolic blood pressure component.

In some implementations, the blood pressure device 182 is a sphygmomanometer including an inflatable cuff that can be worn by a user and a pressure sensor (e.g., the pressure sensor 132 described herein). For example, as shown in the example of FIG. 2, the blood pressure device 182 can be worn on an upper arm of the user 210. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff. More generally, the blood pressure device 182 can be communicatively coupled with, and/or physically integrated in (e.g., within a housing), the control system 110, the memory 114, the respiratory therapy system 120, the user device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation level (SpO₂), electrodermal activity (also known as skin conductance or galvanic skin response), a position of the user, a posture of the user, or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory 114, the respiratory therapy system 120, and/or the user device 170, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user's use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, the user device 170, and the blood pressure device 182 and/or activity tracker 190. As yet another example, a third alternative system includes the control system 110, the memory device 114, the respiratory therapy system 120, at least one of the one or more sensors 130, and the user device 170. As a further example, a fourth alternative system includes the control system 110, the memory device 114, the respiratory therapy system 120, at least one of the one or more sensors 130, the user device 170, and the blood pressure device 182 and/or activity tracker 190. Thus, various systems can be formed using any portion or portions of the components shown and described herein and/or in combination with one or more other components.

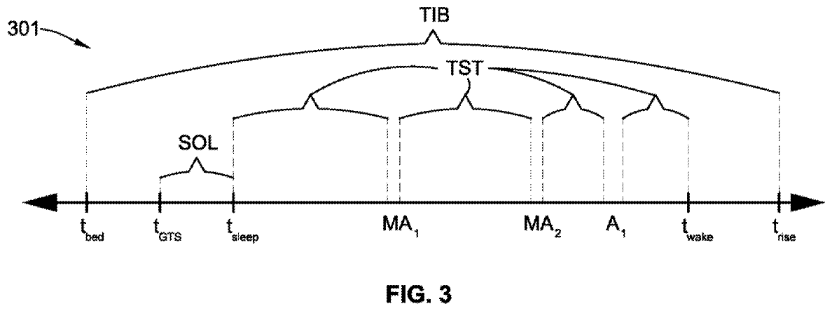

Referring to the timeline 301 in FIG. 3, the enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 4 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 3 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 4 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

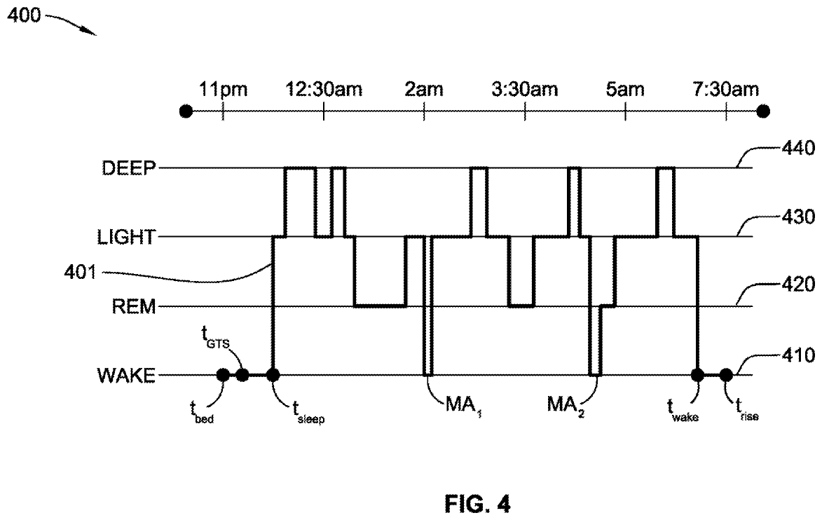

Referring to FIG. 4, an exemplary hypnogram 400 corresponding to the timeline 301 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 4 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM stage) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

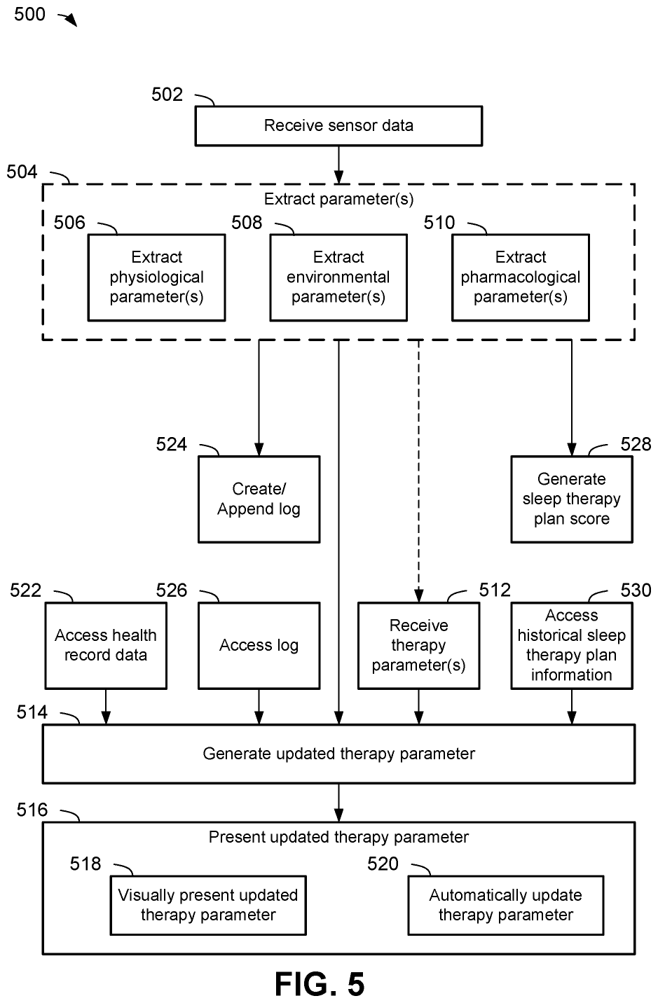

FIGS. 5-7 relate to facilitating engagement of a sleep therapy plan. A sleep therapy plan is a set of instructions, variables, and/or other elements used to define a particular course of sleep therapy for an individual. Sleep therapy can include any set of procedure(s) that a user follows to treat a sleep-related condition. As used herein, the term sleep therapy is generally intended to refer to treatment of a sleep-related condition using means other than respiratory therapy. Certain aspects of the present disclosure are especially useful for facilitating engagement of a plan for following behavioral sleep therapy (e.g., sleep therapy that involves monitoring, adjusting, or otherwise dealing with an individual's behavior). An example of behavioral sleep therapy is CBTi. In some cases, sleep therapy can include a combination of behavioral sleep therapy and another type of sleep therapy (e.g., a pharmacological intervention program, such as sleep aids like antihistamines, hypnotics, etc.). In some cases, the another type of sleep therapy may include a Sleep Disordered Breathing (e.g., sleep apnea) therapy such as PAP, MRD, etc.

CBTi is a type of behavioral sleep therapy that involves multiple components designed to treat insomnia. Each CBTi component can include instructions and strategies for monitoring and modifying behavior to treat aspects of insomnia. In a stimulus control component, an individual can take various actions to strengthen the individual's association between bed and sleeping. In a sleep restriction component, sleep quality is targeted at the expense of sleep quantity by purposefully limiting the amount of time spent in bed and only increasing it stepwise after sleep quality has sufficiently improved. In a sleep-interfering arousal/activation component, techniques are used to manage stress, thoughts, and the like to help limit the presence of sleep-interfering thoughts. CBTi can also include components to help promote certain eating habits (e.g., limiting certain substances, such as alcohol and stimulants, prior to sleeping), reinforce the user's biological clock (e.g., by matching bed times to the circadian clock), and the like. An important aspect of CBTi is the collection of log data and occasional meetings with healthcare professionals to evaluate the log data and make alterations to the CBTi plan going forward.

A sleep therapy plan can be defined by a set of therapy parameters and/or instructions for implementing the therapy parameters. Any number and type of therapy parameters can be used to describe any given sleep therapy plan. For example, a CBTi sleep therapy plan can include many therapy parameters, such as i) a target in-bed time; ii) a target out-of-bed time; iii) a target sleep time (e.g., a time when sleep is to begin); iv) a target awaken time (e.g., a time when the individual should awaken); v) an alarm time (e.g., a time when an alarm should go off to awaken the individual); vi) a target sleep duration (e.g., a TST or PTST); vii) a pharmacological dosage parameter (e.g., a parameter denoting a general category of medication, a specific medication, a target quantity of medication, a target time of using the medication, information about how the medication is to be used, information about what is to be performed or avoided before or after using the medication, or any other pharmacology-related information associated with the user); viii) a sleep environment parameter (e.g., a light level in the environment, a temperature level in the environment, a sound level in the environment, a category of environment, or any other environment-related information associated with the user); ix) a pre-sleep activity parameter (e.g., a list of one or more activities to perform or avoid before a sleep session or before falling asleep, a target amount of time that should be left after exercising and before starting a sleep session, a target relaxation exercise to perform; or any other information related to activities an individual may perform prior to a sleep session); or x) any combination of i-ix. A CBTi sleep therapy plan can include other therapy parameters as well.

Certain aspects and features of the present disclosure relate to using sensor data (e.g., passive and/or active acoustic sensing, RADAR sensing (using e.g., using FMCW or CW signals), and the like to measure biomotion in a non-contacting fashion) to pre-screen an individual for sleep-related disorders that may affect the efficacy of a sleep therapy plan (e.g., a CBTi sleep therapy plan) and/or otherwise endanger the individual. Certain aspects and features of the present disclosure relate to using sensor data (e.g., via extracted physiological parameters) to intelligently pre-configured and/or update therapy parameters of a sleep therapy plan (e.g., a CBTi sleep therapy plan), such as pre-configuring in advance and/or updating in realtime or near realtime. Certain aspects and features of the present disclosure relate to using sensor data to automatically generate (e.g., create and/or append) logs associated with a sleep therapy plan (e.g., a CBTi sleep therapy plan) to reduce burden on an individual engaging in a sleep therapy plan. Certain aspects and features of the present disclosure relate improving efficacy of a sleep therapy plan (e.g., a CBTi sleep therapy plan) by automatically monitoring, logging, and/or acting in response to detected stimuli or actions that are discouraged by the sleep therapy plan (e.g., notifying the user when they are using a smartphone or watching television at times when they should not be doing so according to their sleep therapy plan). Certain aspects of the present disclosure may be combined with respiratory therapy, although that need not always be the case.

Certain aspects and features of the present disclosure can identify insomniac candidates, including insomniac candidates who may benefit from a sleep therapy plan, such as CBTi. Certain aspects and features of the present disclosure can identify physiological parameters, such as anxiety and stress, which may be causing insomnia, via requesting subjective feedback (e.g., providing a questionnaire) and/or sensor data (e.g., detecting hyperarousal from heart rate changes). Certain aspects and features of the present disclosure can collect sensor data only i) during a sleep session; ii) during and adjacent to (e.g., shortly (e.g. within about 15, 30 or 60 minutes) before or after) a sleep session; iii) during times other than the times during and adjacent to the sleep session; or iv) any combination of i-iii. Certain aspects and features of the present disclosure collect sensor data using only i) non-contact sensors; ii) wearable sensors; iii) respiratory therapy device sensors; or iv) any combination of i-iii. Certain aspects and features of the present disclosure facilitate engaging in certain sleep therapy plans, such as a CBTi sleep therapy plan, by using sensor data as disclosed herein as an alternative to some or all of manual questionnaires and manual data logging.

In some cases, certain aspects of the present disclosure can be performed prior to implementation of a sleep therapy plan, such as to pre-screen an individual for sleep therapy (e.g., a user with SDB such as OSA may be incompatible with CBTi or may require adjustment of the CBTi program) and/or obtain baseline data. In some cases, certain aspects of the present disclosure can be performed while the user is engaging in a sleep therapy plan, which can include while the user is in a sleep session or between sleep sessions while the user is still in the course of a sleep therapy plan, such as to automatically adjust therapy parameters or monitor efficacy of the current sleep therapy plan. In some cases, certain aspects of the present disclosure can be performed after completion of a sleep therapy plan, such as to monitor efficacy of the completed sleep therapy plan and/or pre-screen for a future sleep therapy plan (e.g., in the case of potential insomnia relapse, in which case all, some, or none of the past sleep therapy plan can be restarted or continued).

In a first example use case, a user may have a smartphone app that uses non-contact sensors (e.g., microphone and speakers of the smartphone in the form of, for example, an active acoustic (sonar) and/or passive acoustic sensor) to detect biomotion of the user during sleep and provide an analysis of the user's sleep session. In this use case, the smartphone app may identify that the user is exhibiting signs of SDB such as OSA (e.g., due to detected apneas or other sleep events). At that time or a later time, the smartphone app may detect that the user is exhibiting signs of insomnia. The smartphone app may provide a recommendation to the individual to have their insomnia treated, but may warn against certain sleep therapy plans or certain components of certain sleep therapy plans. In this example, the recommendation may include a recommendation that the user seek out a professional to assist with CBTi, along with a warning that it may be advisable to avoid the sleep restriction aspects of CBTi. In some cases, the smartphone app may automatically adjust a CBTi program and/or may help implement an alternative CBTi program that is compatible with the user's SDB such as OSA.

In an example, if a user exhibits an AHI of at or greater than 5 for one or more nights, there is a significant risk that sleep restriction in CBTi could lead to profound sleepiness and potential accident the following day. Thus, a CBTi program may need to be adjusted and/or avoided for certain individuals with SDB (e.g., OSA).

In a second example use case, a user may have a smartphone app that uses non-contact sensors (e.g., microphone and speakers of the smartphone) to detect biomotion of the user during sleep and provide an analysis of the user's sleep session. In this use case, the smartphone app may identify that the user is exhibiting signs of OSA (e.g., due to detected apneas or other sleep events) and may identify that the user appears to be engaging in certain actions indicative of the user practicing a sleep therapy plan, such as a CBTi plan. The smartphone app, optionally after presenting a confirmation to the user (e.g., “Are you currently using a CBTi plan or engaging in intentional sleep restriction?”), may present a warning to the user that a CBTi plan or sleep restriction may be discouraged because it appears the user has OSA.

In a third example use case, a user may have a smartphone app that uses non-contact sensors (e.g., microphone and speakers of the smartphone) to detect biomotion of the user during sleep and provide an analysis of the user's sleep session. In this use case, the user may be undergoing sleep therapy, such as a sleep restriction component of a CBTi plan. Instead of merely setting a static alarm for a given time (e.g., 5 am after going to bed at around 11:30 pm), the user may simply set a target sleep duration. The smartphone app will then use the detected biomotion to identify when the user has fallen asleep, then automatically trigger the alarm to go off after the user has achieved the target sleep duration, optionally while the user is in a particular sleep stage or set of sleep stages. Additionally, the smartphone app can generate a log of sleep-related data for use with the CBTi plan.

In a fourth example use case, a user may have a smartphone app that uses non-contact sensors (e.g., microphone and speakers of the smartphone) to detect biomotion of the user during sleep and provide an analysis of the user's sleep session. In this use case, the user may be undergoing sleep therapy, such as a sleep-interfering arousal/activation component of a CBTi plan. The smartphone app may use the detected biomotion or other sensor data to detect that the user is preparing to go to sleep. The smartphone app may also detect one or more sleep-interfering elements, such as use of the smartphone or a given app on a smartphone, elevated light levels in the bedroom, elevated sound levels in the bedroom, use of a television, or the like. The smartphone app may then provide a notice to the user (e.g., “It appears you may be watching TV. Your CBTi plan recommends not watching TV within 30 minutes of going to bed.”) and/or automatically take action to remove or reduce the sleep-interfering element (e.g., automatically adjusting the light level or sound level of one or more devices in the environment).

In a fifth example use case, a user may have a smartphone app that uses non-contact sensors (e.g., microphone and speakers of the smartphone) to detect biomotion of the user during sleep and provide an analysis of the user's sleep session. In this use case, the user may be undergoing sleep therapy. The smartphone app may detect that the user has taken a nap earlier in the day. As a result, the smartphone app may automatically adjust one or more parameters of the sleep therapy plan (e.g., adjusting the target in-bed time of a CBTi plan) based on the user's nap.

FIG. 5 is a flowchart depicting a process 500 for updating a sleep therapy plan according to some implementations of the present disclosure. Process 500 can be performed by system 100 of FIG. 1, such as by a user device (e.g., user device 170 of FIG. 1). Process 500 can be performed in realtime or near realtime.

At block 502, sensor data is received. The sensor data can be received from one or more sensors, such as one or more sensors 130 of FIG. 1. The sensor data received at block 502 can be biometric sensor data, although that need not always be the case. The received sensor data can include any suitable sensor data as disclosed herein, including, for example, heart rate data, individual temperature data, movement data, biomotion data, environmental light data, environmental temperature data, pharmacological data and the like. In some cases, sensor data from one or more sensors can be used to synchronize additional sensor data from one or more additional sensors. In some cases, parameters identified from one or more channels of sensor data at block 504 can be used to help synchronize the channels of sensor data. As noted herein, the sensor data may be generated by i) one or more non-contact sensors (such as passive and/or active acoustic sensor, a radar sensor, etc.); ii) one or more wearable sensors (such as smartwatches with medical grade (e.g., FDA-approved) physiological sensors); iii) one or more respiratory therapy device sensors (such as a flow sensor, a pressure sensor, a microphone, etc.); or iv) any combination of i-iii. In an example, sensor data may be generated by a non-contact sensor (such as a passive and/or active acoustic sensor) and a wearable sensor (such as a PPG sensor, ECG sensor, which may be mounted in a smartwatch or a fingertip probe). In another example, sensor data may be generated by a non-contact sensor (such as a passive and/or active acoustic sensor) and a respiratory therapy device sensor (such as a flow sensor and/or a pressure sensor).

In some cases, the sensor data specifically includes biomotion data, such as biomotion data acquired via one or more non-contact sensors as disclosed herein. Biomotion data can relate to movement of the user due to respiration and/or gross bodily movements (such as limb movements before, during and/or after a sleep session. In some cases, the use of non-contact sensors can be especially important since the user is suffering from insomnia, in which case a contacting sensor may further interfere with the user's ability to sleep. Such a sensor (e.g., a passive and/or active acoustic sensor; a radar sensor; or a remote PPG sensor, as described herein) may be placed at a bedside and, once active, detect the presence/absence of the user and, once presence is detected, begin generating biomotion data associated with the user without the user's further input or interaction with the sensor. As mentioned, biomotion data can include information related to body movement, which can include movement of any part of a user's body, such as the user's chest, the user's arms, the user's legs, and the like. In some cases, body movement information includes respiration-related movement information.

At block 504, one or more parameters can be extracted from the received sensor data. Extracting parameters can include extracting one or more physiological parameters at block 506, one or more environmental parameters 508, one or more pharmacological parameters 510, or other suitable parameters. In some cases, a parameter can be based on one or more other parameters (e.g., one or more parameters can serve as a basis for another parameter). In some cases, a parameter can be a change between two parameters, such as a rate of change or an amount of change.

At block 506, extracting physiological parameters can include processing the received sensor data and extracting physiological parameters associated with the user, such as heart rate, heart rate variability, temperature of the individual (e.g., skin temperature), temperature variability, respiration rate, respiration rate variability, breath morphology, EEG activity, EMG activity, ECG data, and the like.

In some cases, a physiological parameter can be a sleep-related parameter, although in some cases a sleep-related parameter can be a non-physiological parameter. A sleep-related parameter is a parameter associated with a sleep session of a user. Examples of sleep-related parameters that are physiological parameters include an Apnea-Hypopnea Index (AHI) score, a sleep score, a respiration signal, a respiration rate, an inspiration amplitude, an expiration amplitude, an inspiration-expiration ratio, a number of events per hour, a pattern of events, a sleep stage, a heart rate, a heart rate variability, movement of the user, temperature, EEG activity, EMG activity, arousal, snoring, choking, coughing, whistling, wheezing, or any combination thereof. Examples of non-physiological sleep-related parameters include parameters associated with a respiratory therapy device, such as flow or pressure settings of a respiratory device, among others. In some cases, parameters extracted from sensor data received from a respiratory therapy device can be useful in extracting physiological parameters or other parameters.

In some cases, knowledge of a sleep stage information can be especially useful when a user is engaging in sleep restriction. During sleep restriction, a user may experience an unusually high ratio of deep sleep to REM sleep. During sleep restriction there may be a rebound effect where the AHI would increase significantly during REM and provide an artificially high AHI. Therefore, artificially high AHI can be accounted for by having knowledge of sleep stage information along with knowledge of one or more therapy parameters (e.g., therapy parameters from block 512, such as sleep restriction parameters).

In some cases, extracting physiological parameters can be based on biomotion sensor data. Biomotion information can be extracted from biometric sensor data. Chest movement information can be extracted from the biomotion information by processing the biomotion information. Various physiological parameters, including sleep-related parameters, can be determined by processing the chest movement information, such as, for example, Apnea-Hypopnea Index (AHI) score, a sleep score, a respiration signal, a respiration rate, an inspiration amplitude, an expiration amplitude, an inspiration-expiration ratio, a number of events per hour, a pattern of events, and a sleep state and/or sleep stage. In some cases, the biomotion sensor data can be acquired from non-contact sensors.

In terms of determining physiological parameters associated with tracking time in bed (TiB) versus sleep efficiency, such tracking can be age dependent. For a middle aged cohort, exemplar parameters might be if sleep efficiency is >=90%, the target sleep duration should be increased; if the sleep efficiency is 85-90%, no change should be made to target sleep duration; and if the sleep efficiency is dropping below 85%, which suggest excessive time awake when the user is trying to sleep, then the target sleep duration should be decreased (e.g., by 15-30 minutes). These changes could be effected by adjusting therapy parameters to wake the person early and/or delaying the target bed time for the next sleep session-and, for example, noting that no naps should be taken (unless safety critical).

Extracting environmental parameter(s) at block 508 can include extracting information about the environment from the received sensor data from block 502. Environmental parameter(s) can include any parameter associated with the environment in which the user is situated and/or in which the user engages in a sleep session. Examples of suitable environmental parameter(s) include environment temperature, humidity, noise level, light level, and the like.

In some cases, noise in the environment can be identified. Such noises may relate to behavioral or non-behavioral sources. For example, background noise or a snoring bed partner may be keeping the user awake and/or waking them during a sleep session. In some cases, noise may be related to the user's movements, such as if the bed or bed frame is noisy. In some cases, environmental lighting conditions, such as the light level, can be detected. Light level can be used to adjust a targeted (recommended) light level for sleeping according to the sleep therapy plan, which can be achieved by the user making changes (e.g., closing curtains) or via automatic control (e.g., by adjusting smart light bulbs or automatically shutting motorized blinds). Similarly, environmental parameter(s) related to the temperature of the sleeping environment can be detected and used to adjust a targeted (recommended) temperature for sleeping according to the sleep therapy plan.

Extracting pharmacological parameters at block 510 can include extracting information about one or more medications taken or explicitly not taken when expected by the user. For example, pharmacological parameter(s) can include a general category of medication, a specific medication, a quantity of medication, a time of using the medication, information about how the medication is used (e.g., how the medication is taken by the user, such as with or without water), information about what is to be performed or avoided before or after using the medication (e.g., whether or not the user avoided eating food after taking the medication), or any other pharmacology-related information associated with the user.

As an example, a user taking sleep aids can be monitored as part of a sleep therapy plan. Extracted pharmacological parameter(s) can be used to log medication dosing, log skipped medication, and the like. If a medication is found to be taken and/or skipped, the system can automatically update therapy parameters accordingly, such as to provide a greater or smaller sleep duration when a sleep aid is determined to have been taken by the user.

As used herein, one or more parameter(s) extracted at block 504 can serve as a basis for a subsequent block to be based at least in part on sensor data from block 502. For example, a log that is based at least in part on sensor data from block 502 can be based at least in part on the sensor data via one or more parameter(s) extracted at block 504.

At block 512, one or more therapy parameters are received. Receiving a therapy parameter can include accessing the therapy parameters stored locally (e.g., stored in local memory), accessing the therapy parameter stored on an external source (e.g., a remote medical database), obtaining the therapy parameter from user input (e.g., via a user questionnaire), predicting the therapy parameter based on one or more extracted parameters from block 504, or otherwise. Receiving the one or more therapy parameters at block 512 can include determining that the user is engaging in a sleep therapy plan. In some cases, receiving the one or more therapy parameters at block 512 can include determining that the user is engaging in a sleep therapy plan that is CBTi or that includes one or more components of CBTi, such as sleep restriction.

Any suitable therapy parameters can be received at block 512. In an example, a target sleep duration is received at block 512. The target sleep duration can be a length of time the user is planning to sleep during the next sleep session as outlined in their sleep therapy plan, such as five hours. Any other therapy parameter, including those described in further detail herein, can be received at block 512.

At block 514, an updated therapy parameter is generated. Generating the updated therapy parameter can include generating a new therapy parameter that is not currently used in the sleep therapy plan, generating a replacement value for a therapy parameter in use in the sleep therapy plan, or generating an amount of change to be applied to a therapy parameter in use in the sleep therapy plan.

Generating the updated therapy parameter at block 514 can be based on one or more factors, including one or more extracted parameters from block 504. Generating the updated therapy parameter at block 514 can include using i) extracted physiological parameter(s) from block 506; ii) extracted environmental parameter(s) from block 508; iii) extracted pharmacological parameter(s) from block 510; iv) extracted sleep-related parameters otherwise from block 504; or v) any combination of i-iv.

In an example, a physiological parameter extracted at block 506 can include sleep stage information (e.g., sleep stage information as seen with reference to sleep-wake signal 401 of FIG. 4). In an example case, the physiological parameter can be indicative of a time when the user fell asleep. In such an example, process 500 can include generating an updated alarm time based on the time when the user fell asleep (e.g., initial sleep time) and a target sleep duration therapy parameter from block 512. The updated alarm time can be a new therapy parameter (e.g., if no alarm time was previously established for the sleep therapy plan) or an updated therapy parameter (e.g., if a different alarm time was previously established for the sleep therapy plan). As described in further detail herein, the updated alarm time therapy parameter can be used to update a sleep therapy plan, such as in realtime. Thus, the time at which the system will trigger the alarm will be automatically updated based on the user's actual initial sleep time.

In some cases, generating the updated therapy parameter at block 514 can include using multiple factors, such as multiple extracted parameters from block 504. For example, a future alarm time can be based not only on an initial sleep time, but also a total sleep time or persistent total sleep time. For example, with respect to the user from the previous example, further iterations of process 500 can include extracting physiological parameter(s) at block 506 indicative of a number of microawakenings. In such cases, the updated alarm time generated at block 514 can be further based on the information about microawakenings, such as by delaying the updated alarm time by the duration of the microawakening(s). Thus, the system will automatically and dynamically ensure the user obtains the target duration of sleep despite any microawakenings or other awakenings that may occur after initially falling asleep.

In an example, generating the updated therapy parameter at block 514 can include customizing the optimal awakening time to a smart alarm feature that preferentially wakes the user from a light N1 or N2 sleep stage (e.g., as determined from extracted parameter(s) from block 504). In some cases, the optimal awakening time can be customized by dynamically updating the wakeup time (e.g., alarm time) earlier or later to target a sleep efficiency percentage. In some cases, such a targeted sleep efficiency percentage can be weighted by a number of days into a course of sleep therapy plan. For example, a user initially starting a sleep therapy plan may be provided with less onerous targets to ease the user into the sleep therapy plan and to promote a sense of accomplishment to improve compliance with the sleep therapy plan. In some case, the system can target a sleep efficiency percentage that is weighted by the number of days into the program, once the system checks if the user can achieve any sleep efficiency improvement over their own baseline (e.g., a pre-programmed baseline or a detected baseline). In some cases, an awakening time can be customized based on a turning total of the time in bed and sleep efficiency to that point in a sleep session.

In some cases, the system can update certain therapy parameters based on detecting that a user is likely to wake during a sleep session. If the user awakens and the system determines that the user is unlikely to fall asleep quickly (e.g., based on received sensor data), updating the therapy parameter can include updating a new in-bed time and/or updating a new pre-sleep activity therapy parameter to encourage the user to get out of bed and do something until they are tired again. In an example, such an update can be effected by turning on a light in the user's environment. Such actions can deliberately force or strongly suggest that the user get out of bed and only return when they are tired again so they can restart their sleep session while tired.

In some cases, if the user's sleep-related parameters have improved from below a threshold to above a threshold, the system can trigger a pause in particularly onerous CBTi tasks, as an early “success” has been achieved. For example, if the user has sufficiently improved sleep quality during a course of sleep restriction, the system may automatically adjust a therapy parameter to eliminate and/or reduce the sleep restriction since the user may no longer need it.

At block 516, the updated therapy parameter generated at block 514 can be presented. Presenting the updated therapy parameter can include automatically applying the updated therapy parameter, prompting the user before automatically applying the updated therapy parameter or allowing the user to manually apply the updated therapy parameter, or prompting another individual (e.g., healthcare professional) before automatically applying the updated therapy parameter or allowing the another individual to manually apply the updated therapy parameter. Automatic application of the updated therapy parameter can occur in realtime or near realtime (e.g., dynamically changing a therapy parameter as the user sleeps), or delayed (e.g., changing a therapy parameter between sleep sessions).

In some cases, presenting the updated therapy parameter can include visually presenting the updated therapy parameter to the user at block 518. Visually presenting the updated therapy parameter can include presenting to the user, such as via a display device or otherwise, an indication that a particular therapy parameter should be changed to achieve a more desirable result. In some cases, visually presenting the updated therapy parameter can include presenting information to facilitate the user making the change to the sleep therapy plan (e.g., instructions about how to enact the change).

In some cases, visually presenting the updated therapy parameter at block 518 can include presenting the updated therapy parameter to an individual other than the user, such as a healthcare professional or other caregiver. For example, in some cases, a healthcare provider managing a user's sleep therapy plan may be notified about a suggested change to the user's sleep therapy plan, providing the healthcare provider an opportunity to i) accept the change and automatically implement the change or otherwise facilitate implementation of the change; ii) consider the change for a subsequent follow-up session with the user; or iii) reach out to the user to discuss the change.

In some cases, visually presenting the updated therapy parameter at block 518 can include engaging the user using a chatbot or other such engagement. In some cases, if certain criteria are met, the system can facilitate connection with a person for coaching, such as a healthcare professional.

In some cases, presenting the updated therapy parameter at block 516 can include automatically updating the therapy parameter at block 520. Automatically updating the therapy parameter can include adjusting the therapy parameter of the sleep therapy plan. For example, an alarm time therapy parameter can be automatically adjusted by changing the alarm time.

In some cases, automatically updating a therapy parameter can include automatically effecting a change associated with the therapy parameter. For example, an environmental light therapy parameter that is automatically adjusted from a first setting to a lower (e.g., darker) setting can include automatically adjusting a light source to effect the change and achieve the lower setting. As another example, adjusting an in-bed time can automatically adjust a notification or reminder presented to the user to go to bed.

In some optional cases, process 500 can include creating and/or appending a log at block 524. Creating and/or appending the log at block 524 can include generating one or more log entries based at least in part on one or more extracted parameters from block 504. Any suitable information can be stored in a log, including objective data (e.g., from one or more biological sensors) and subjective data (e.g., from user feedback). Examples of subjective data include an amount of restfulness felt by the user, a level of sleep quality perceived by the user, an in-bed time that the user believes is correct, or the like. In some cases, objective data obtain from sensor data from block 502 can be used to confirm, refute, or adjust subjective data. Use of objective data to compare with subjective data can help a user identify and become more aware of their objective data and its correlation to subjective data. In some cases, if a gap between objective and subjective data is identified and does not converge as expected (e.g., for their demographic), the sleep therapy plan may be adjusted to regress and give the user additional time or opportunities to improve. In some cases, such a gap may trigger a chatbot session or a communication with a healthcare professional. In some cases, the log can contain only subjective data, only objective data, or a combination thereof. Some examples of information stored in a log include i) sleep state information; ii) sleep stage information; iii) sleep efficiency information; iv) sleep quality information; v) an actual in-bed time; vi) an actual out-of-bed time; vii) sleep environment information; viii) detected pre-sleep activity information; or ix) any combination of i-viii.

In some cases, one or more therapy parameters (e.g., therapy parameter(s) received at block 512) can be used to establish what parameters are used to generate a log entry. For example, a therapy parameter of a sleep therapy plan can indicate that the user is to prepare a log (e.g., a sleep diary) tracking the user's in-bed time, sleep onset latency, sleep duration, and out-of-bed time. Using this information, the system can make use of the appropriate parameters extracted at block 504 to create and/or append to the log. In some cases, the log can include raw sensor data and/or extracted parameter(s).

In some optional cases, generating an updated therapy parameter at block 526 can include using log data accessed at block 526. Block 526 can include accessing a historical log, which can be the same log from block 524 or another log (e.g., a pre-existing log). The log can include sleep-related information and/or sleep-therapy-related information. For example, the log may include past in-bed times, past sleep durations, and past sleep scores. In an example, if the system determines that past sleep durations above a threshold are correlated with higher past sleep scores, generating the updated therapy parameter at block 514 can include increasing a current target sleep duration therapy parameter (e.g., from block 512) that is below the threshold to a value that is above the threshold.

In some optional cases, generating an updated therapy parameter at block 526 can include accessing health record data at block 522. Accessing health record data can include accessing health record data from the user (e.g., via a questionnaire) or from a remote source (e.g., a medical records database). Health records can include medical information about the user, including diagnoses, suspected diagnoses, medication, medical history, and the like. Such health record data can be used to generate an updated therapy parameter. For example, knowledge of an existing health condition, alone or in combination with one or more extracted parameter(s), may warrant an updated therapy parameter. In such an example, knowledge of the user suffering from post-traumatic stress disorder, anxiety, depression, and/or comorbidity may benefit more from a modified version of sleep restriction (e.g., without as onerous restrictions as would otherwise be used). In some cases, health record data can include information such as untreated OSA or other untreated sleep-related conditions.

For example, early data on insufficient sleep time being allowed for a user can be helpful for the system in updating therapy parameters. In some cases, knowledge of a user's profession (e.g., a shift worker, a worker with a safety critical job, a worker with high risk if attention is low (e.g., a driver)) can be useful in updating therapy parameters. Such information can allow separation of a presumed insufficient sleep time due to scheduling (e.g., not providing opportunity for sleep) versus one due to insomnia.

As another example, fall risk (e.g., from medical records, prior risk of fall, detected gait, physiological parameter(s), and the like) can be a useful input for determining updated therapy parameters. For example, where there is a fall risk, generating an updated therapy parameter at block 514 can generate an updated therapy parameter designed to decrease the severity of any CBTi sleep restriction in order to reduce the change of negative health consequences (e.g., one would not want to trigger a fall with trying to fix behaviors leading to insomnia). Thus, a sleep therapy plan can be automatically adjusted based on a user's risk factors. Consideration of a user's risk factors can be based on data other than health record data, as well. For example, extracted pharmacological parameter(s) can be used to identify when a user may have increased risk factors in the future, and one or more therapy parameters can be adjusted accordingly.

In some optional cases, a sleep therapy plan score can be generated at block 528. The sleep therapy plan score can be generated based at least in part on one or more extracted parameters from block 504. The sleep therapy plan score can be indicative of an efficacy of a sleep therapy plan. In some cases, the sleep therapy plan score can be stored in association with one or more therapy parameters (e.g., therapy parameters from block 512) and/or other sleep therapy plan information (e.g., a category of sleep therapy plan, such as behavioral sleep therapy or CBTi). The sleep therapy plan score can be based on parameters indicative of a quality of sleep, a duration of sleep, a subjective feeling, an in-bed time, or other parameters or any combination of parameters. For example, for a user desiring to fall asleep within 30 minutes of an in-bed time and sleep at least 6 hours each night, the system can generate a sleep therapy plan score based on an in-bed time, an initial sleep time, a sleep duration, and optionally sleep stage information. If the user achieves their targets, the sleep therapy plan score may be high (e.g., 100 out of 100). If the user is not yet close to achieving their targets, the sleep therapy plan score may be low (e.g., 20 out of 100). Associating a sleep therapy plan score with therapy parameters and/or other sleep therapy plan information can allow the system to identify therapy parameters or other aspects that are more likely than others or otherwise are expected to improve the user's sleep.

In some optional cases, generating an updated therapy parameter at block 530 can include accessing historical sleep therapy plan information. Historical sleep therapy plan information can include historical sleep therapy plan scores (e.g., sleep therapy plan scores generated in previous iterations of block 528) as well as other information associated with a sleep therapy plan. Information from previous sleep therapy plans that were attempted by the user can be used to inform how one or more therapy parameters will be updated at block 514. For example, if changing the in-bed time has had little to no effect on the user's sleep during previous courses of sleep therapy, the system may opt to change one or more other therapy parameters other than in-bed time.

The historical sleep therapy plan information can be received from local or remote data sources. In some cases, historical sleep therapy plan information can include historical therapy parameters, historical sensor data, historical parameters (e.g., historical physiological parameters, environmental parameters, and/or pharmacological parameters), and the like. Historical sleep therapy plan information can include knowledge of past sleep therapy plans (e.g., past therapy parameters) in which the user has previously engaged (individualized historical sleep therapy plan information) or in which other users having similar demographic information have previously engaged (demographic historical sleep therapy plan information).

In some cases, process 500 can repeat by continuing to receive sensor data at block 502. Process 500 can repeat daily, weekly, monthly, or at other rates. In some cases, process 500 repeats in realtime or near realtime (e.g., at a sampling rate at or under 3 hours, 1 hour, 45 minutes, 30 minutes, 15 minutes, 10 minutes, 5 minutes, 1 minute, 30 seconds, 15 second, 10 second, 5 seconds, or 1 second). While the blocks of process 500 are depicted in a certain order, some blocks can be removed, new blocks can be added, and/or blocks can be moved around and performed in other orders, as appropriate. Additionally, while not always depicted, in some cases one or more blocks may use, as an input, an output of one or more other blocks. For example, in some cases, creating/appending a log at block 524 may use a received therapy parameter from block 512 as an input.

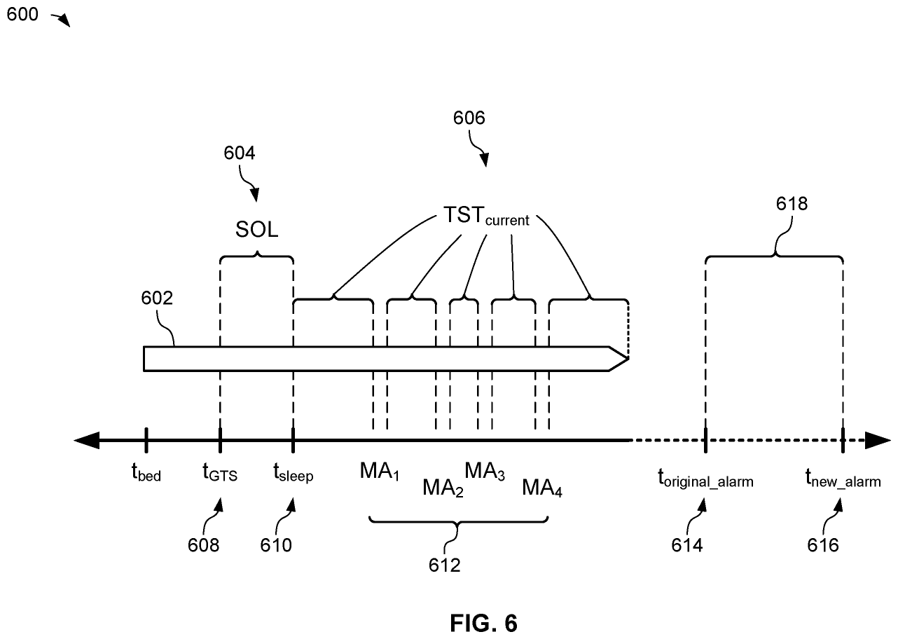

FIG. 6 is a timeline diagram 600 depicting dynamic updating of a sleep therapy plan during a sleep session, according to some implementations of the present disclosure. The timeline diagram 600 of FIG. 6 can be an example of a timeline similar to that of FIG. 3, although while the user is still engaging in the sleep session and before the sleep session has concluded. Timeline diagram 600 can represent an implementation of process 500 of FIG. 5. Arrow 602 is indicative of the user's progress through the sleep session, as monitored by received sensor signals (via one or more extracted parameters, such as physiological parameters). In the timeline diagram 600, t_{bed} is indicative of the time the user went to bed, t_{GTS} 608 is indicative of when the user initially attempts to go to sleep, tₛₗₑₑₚ 610 is indicative of the time when the user initially falls asleep, microawakenings 612 (e.g., MA₁, MA₂, MA₃, and MA₄) are indicative of microawakenings in which the user is not fully asleep, t_{original_alarm} 614 is indicative of an original value for an alarm time therapy parameter for the sleep therapy plan, and t_{new_alarm} 616 is indicative of a new (e.g., updated) value for the alarm time therapy parameter. SOL 604 indicates the sleep-onset-latency time, or length of time between when the user attempts to go to sleep and when the user initially falls asleep. TST_{current} 606 is indicative of the current total sleep time of the user. The final TST for the user will be TST_{current} plus any additional time the user spends asleep before ultimately waking.

At the current moment in time depicted by timeline diagram 600, the user is sleeping after having fallen asleep at tₛₗₑₑₚ 610. Based on the original sleep therapy plan, the user is to be awakened by an alarm triggering at time t_{original_alarm} 614. However, the original sleep therapy plan may be based on a target t_{bed}, t_{GTS}, tₛₗₑₑₚ, or expected TST that might be different from the user's actual t_{bed}, t_{GTS}, tₛₗₑₑₚ, or expected TST given TST_{current} and t_{original_alarm}. As differences are identified, the system can automatically adjust the alarm time by moving the alarm time from t_{original_alarm} 614 to t_{new_alarm} 616. The change in alarm time 618 can be due to multiple factors. In an example, the change in alarm time 618 can be calculated as the cumulative amount of time between an expected time (e.g., a target tₛₗₑₑₚ or an expected TST) and an actual or currently estimated time (e.g., an actual tₛₗₑₑₚ 610 as identified by physiological parameters or an estimated TST based on TST_{current}).

In an example where the sleep therapy plan indicates a total sleep time of 6 hours, if the TST_{current} is 4 hours and only 1 hour remains until t_{original_alarm}, the system may automatically adjust the alarm time to a t_{new_alarm} that is at least 2 hours away (e.g., 2 hours plus any predicted additional microawakening time).

As described herein, therapy parameters other than alarm time can be adjusted and parameters other than those depicted in FIG. 6 can be used to generate the updated therapy parameter.

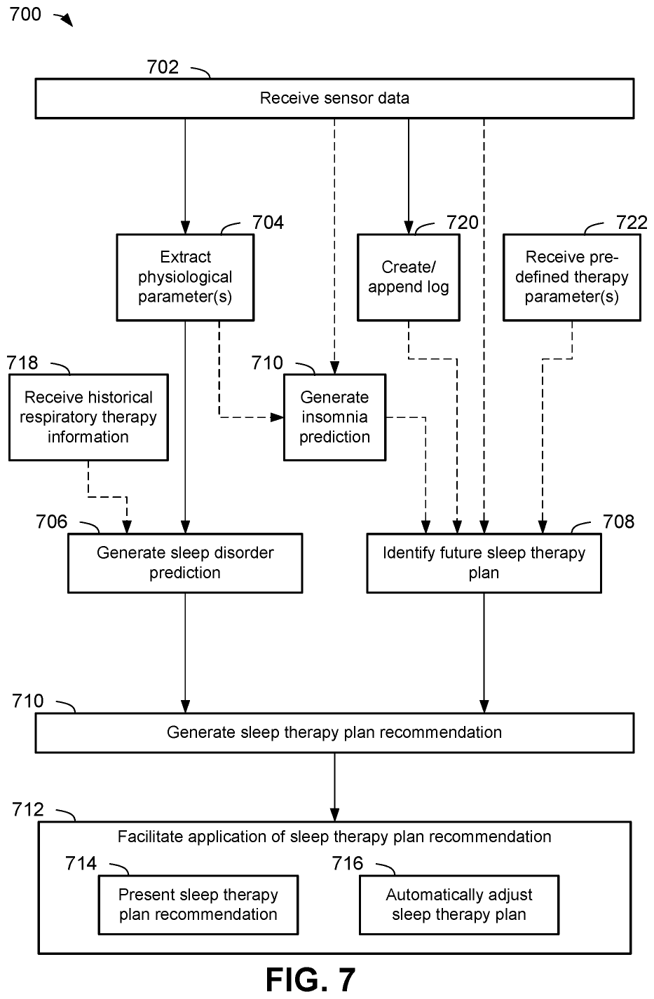

FIG. 7 is a flowchart depicting a process 700 for generating a sleep therapy plan recommendation according to some implementations of the present disclosure. Process 700 can be performed by system 100 of FIG. 1, such as by a user device (e.g., user device 170 of FIG. 1). Process 700 can be performed in realtime or near realtime, although that need not always be the case.

At block 702, sensor data can be received. Sensor data can be received similar to block 502 of FIG. 5. In some cases, sensor data received at block 702 is non-contact sensor data. In some cases, the use of non-contact sensors can be especially important since the user is suffering from insomnia, in which case a contacting sensor may further interfere with the user's ability to sleep.

At block 704, one or more physiological parameters can be extracted. One or more physiological parameters can be extracted similar to block 506 of FIG. 5. In some cases, extracting physiological parameters at block 704 can include detecting one or more sleep events using the received sensor data. Examples of suitable sleep events that can be detected include i) snoring; ii) an apnea event; iii) limb repositioning; iv) body repositioning; v) a sleep state transition; vi) a sleep stage transition; or vii) any combination of i-vi.

In some cases, parameters other than physiological parameters can be extracted in addition to physiological parameters at block 704.

At block 706, a sleep disorder prediction can be generated. Generating a sleep disorder prediction can include using the one or more extracted physiological parameters from block 704. Generating a sleep disorder prediction can include identifying one or more physiological parameters form block 704 that are consistent with and/or indicative of a sleep disorder prediction. For example, an AHI (e.g., calculated by dividing a number of detected apnea and/or hypopnea events during a sleep session by the total number of hours in the sleep session) can be an indicator of sleep apnea as disclosed herein. Combined with oxygen desaturation levels, a severity of OSA can be determined.

In some case, determining a sleep disorder prediction can include generating one or more sleep disorder scores for one or more potential sleep disorders, then determining the sleep disorder prediction based on the one or more sleep disorder scores. For example, if the detected number of sleep events and/or other physiological data is strongly indicative that the user may suffer from OSA, a corresponding sleep disorder score for OSA may be high. If that sleep disorder score for OSA is above a threshold number, the sleep disorder prediction generated at block 706 can be indicative that the user may suffer from OSA.

In an example, biomotion information from extracted physiological parameters from block 704 can be used to detect and identify patterns consistent with PLM(s) (periodic leg movement(s)). In some cases, the physiological parameters can be processed to identify further detail, such as whether the user's PLM is related to unrefreshed sleep or a problem with falling asleep or staying asleep; whether the periodic movements are associated with awakenings; whether treatment may be required; whether it is PLMD; and the like.

In some optional cases, generating a sleep disorder prediction at block 706 can include using received historical respiratory therapy information from block 718. The historical respiratory therapy information can include one or more historical parameters associated with use of a respiratory therapy device. Thus, information collected in association with a user's past use of a respiratory therapy device can be leveraged to generate the sleep disorder prediction.

At block 708, a future sleep therapy plan can be identified. Identifying a future sleep therapy plan can include identifying one or more therapy parameters associated with the future sleep therapy plan. For example, one or more sleep duration parameters can be identified. Sleep duration parameters can include any therapy parameters usable to determine a sleep duration, such as a sleep duration parameter, start and stop sleep time parameters, alarm parameters, and the like.

In some cases, identifying a future sleep therapy plan can include directly receiving sleep therapy plan information, such as therapy parameters associated with the future sleep therapy plan. An example of such a case is a user filling out a questionnaire indicating the intention to engage in a future sleep therapy plan.

In some cases, identifying a future sleep therapy plan can include using received pre-defined therapy parameter(s) from block 722. At block 722, pre-defined therapy parameter(s) can be received, such as from a remote database or the like. A pre-defined therapy parameter includes a therapy parameter that has already been established for the future sleep therapy plan, such as by a healthcare provider treating the user. The healthcare provider can provide the pre-defined therapy parameter at block 722. Thus, when the future sleep therapy plan is identified at block 708, it can be identified based on the received pre-defined therapy parameter(s).

In some optional cases, a log can be created and/or appended at block 720. Creating and/or appending a log at block 720 can be similar to creating and/or appending a log at block 524 of FIG. 5. The log can be created/appended using sensor data and/or extracted parameters (e.g., extracted physiological parameters from block 704). The log can be a sleep quality log. The log can include i) sleep state information; ii) sleep stage information; or iii) a combination of i and ii.

In some cases, identifying a future sleep therapy plan can include using log data, such as sleep quality log data from block 720. The log data can include sleep quality information usable to identify a possible future sleep therapy plan. For example, certain decreases in sleep quality over a period of time may be indicative of a need for sleep therapy, such as behavioral therapy, such as CBTi. In some cases, identifying the future sleep therapy plan can include generating a prediction of a future sleep therapy plan that the user may desire to use.

In some cases, identifying a future sleep therapy plan at block 708 can be based at least in part on extracted physiological parameter(s) from block 704. Sleep quality information and other physiological parameters from block 704 can be indicative of a need for future sleep therapy, such as behavioral therapy, such as CBTi. In some cases, extracted physiological parameter(s) can be indicative that the user is currently engaging in a sleep therapy plan and identifying the future sleep therapy plan can include assuming that the user will continue engaging in the same or a similar sleep therapy plan. In some cases, identifying a future sleep therapy plan based at least in part on extracted physiological parameter(s) can be performed via an insomnia prediction.

At optional block 710, an insomnia prediction can be generated. Generation of an insomnia prediction at block 710 can be based at least in part on received sensor data, such as raw sensor data or via extracted parameters (e.g., extracted physiological parameters from block 704). Generating the insomnia prediction can include identifying sensor data and/or parameters that are characteristic of insomnia. For example, certain in-bed times, sleep onset latency times, and sleep durations can be indicative of insomnia. Once an insomnia prediction is generated, it can be used to identify a future sleep therapy plan at block 708. For example, an indication that the user is likely suffering from insomnia can be an indicator that the user may benefit from sleep therapy, and thus a possible future sleep therapy plan can be identified.

In some cases, generating an insomnia prediction can include generating a stress score based at least in part on the sensor data. The stress score can be indicative of a stress level of the user, which can be used to identify the future sleep therapy plan. The stress level can be identified from objective data (e.g., physiological parameter(s) such as heart rate variability) and/or subjective data (e.g., user response to a questionnaire).

Once a sleep disorder prediction is generated at block 706 and a future sleep therapy plan (e.g., a possible future sleep therapy plan) is identified at block 708, a sleep therapy plan recommendation can be generated at block 710. The sleep therapy plan recommendation is based on the sleep disorder prediction and the future sleep therapy plan. In some cases, the recommendation can be a recommendation or warning regarding engaging in the sleep therapy plan or one or more components of the sleep therapy plan. For example, upon generating a sleep disorder prediction that the user is likely suffering from OSA and identifying a future sleep therapy plan that is CBTi or a similar sleep therapy plan, the sleep therapy plan recommendation generated at block 710 may be a recommendation to avoid sleep restriction aspects of the CBTi plan due to complications that may arise from the user's likely OSA. In some cases, the recommendation can be one or more recommended therapy parameters for a future sleep therapy plan.

In an example, where the system detects possible SDB, such as OSA or CSA, CBTi in of itself may be of little value in treating daytime sleepiness. However, CBTi may help the user fall asleep and reduce time in bed, especially for those with OSA who tend to stay in bed longer. However, treatment with CBTi should be swiftly followed up with PAP or other SDB therapy, as CBTi cannot fix apneas (although side effects of CBTi may temporarily reduce severity of symptoms in some cases, such as due to a better sleep schedule, reduced alcohol content, a better pillow, and the like). The system can thus use knowledge of a predicted sleep disorder and knowledge of the future sleep therapy plan to provide insight, as a sleep therapy plan recommendation, into how to best treat the user's conditions.

In another example, if the system detects possible insomnia due to insufficient sleep syndrome, which is a voluntary disorder based on the user not spending sufficient time in bed, certain sleep therapy plan aspects (e.g., sleep restriction of CBTi) would be ineffective. Thus, the sleep therapy plan recommendation may indicate that certain aspects of the sleep therapy plan are not advised and that the user should focus on treating the insufficient sleep syndrome.

At block 712, application of the sleep therapy plan recommendation can be facilitated. Facilitating application of the sleep therapy plan can include presenting the sleep therapy plan recommendation at block 714 or automatically adjusting a sleep therapy plan at block 716. Presenting the sleep therapy plan recommendation at block 714 can include issuing the recommendation (e.g., warning) to the user, such as via a display device. Presenting the sleep therapy plan recommendation can allow a user to make decisions about how to apply the recommendation, such as by making changes to their sleep therapy plan or discussing such changes with their healthcare provider.

In some cases, presenting the sleep therapy plan recommendation at block 714 can include engaging the user using a chatbot or other such engagement. In some cases, if certain criteria are met, the system can facilitate connection with a person for coaching, such as a healthcare professional.

Automatically adjusting a sleep therapy plan at block 716 can include using the sleep therapy plan recommendation to automatically make changes to a future sleep therapy plan. Making changes to a sleep therapy plan can be similar to updating a therapy parameter as disclosed with reference to process 500 of FIG. 5. In an example, if the sleep disorder prediction indicates that the user is likely suffering from OSA and the future sleep therapy plan is identified as CBTi, automatically adjusting the sleep therapy plan at block 716 can include automatically disabling or adjusting therapy parameters associated with sleep restriction, such as to make sleep restriction less onerous.

In some cases, process 700 can repeat by continuing to receive sensor data at block 702. Process 700 can repeat daily, weekly, monthly, or at other rates. In some cases, process 700 repeats in realtime or near realtime (e.g., at a sampling rate at or under 3 hours, 1 hour, 45 minutes, 30 minutes, 15 minutes, 10 minutes, 7 minutes, 1 minute, 30 seconds, 15 second, 10 second, 7 seconds, or 1 second). While the blocks of process 700 are depicted in a certain order, some blocks can be removed, new blocks can be added, and/or blocks can be moved around and performed in other orders, as appropriate. Additionally, while not always depicted, in some cases one or more blocks may use, as an input, an output of one or more other blocks. For example, in some cases, creating/appending a log at block 720 may use extracted physiological parameter(s) from block 704.

One or more elements or aspects or steps, or any portion(s) thereof, from one or more of any of claims 1 to 52 below can be combined with one or more elements or aspects or steps, or any portion(s) thereof, from one or more of any of the other claims 1 to 52 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method, comprising:
receiving sensor data from one or more sensors, the sensor data being associated with a user engaging in a sleep therapy plan;
receiving one or more therapy parameters associated with the sleep therapy plan;
dynamically generating at least one updated therapy parameter associated with the sleep therapy plan based at least in part on the one or more therapy parameters and the received sensor data; and
presenting the at least one updated therapy parameter in association with the sleep therapy plan.

2. The method of claim 1, wherein presenting the at least one updated therapy parameter includes automatically updating the sleep therapy plan based at least in part on the at least one updated therapy parameter.

3. The method of claim 1, wherein;
the one or more therapy parameters associated with the sleep therapy plan include i) a target in-bed time; ii) a target out-of-bed time; iii) a target sleep time; iv) a target awaken time; v) an alarm time; vi) a target sleep duration; vii) a pharmacological dosage parameter; viii) a sleep environment parameter; ix) a pre-sleep activity parameter; or x) any combination of i-ix; and
wherein the at least one updated therapy parameter includes i) an updated in-bed time; ii) an updated out-of-bed time; iii) an updated target sleep time; iv) an updated target awaken time; v) an updated alarm time; vi) an updated target sleep duration; vii) updated pharmacological dosage parameter; viii) an updated sleep environment parameter; ix) an updated pre-sleep parameter; or x) any combination of i-ix.

4. (canceled)

5. The method of claim 1, wherein receiving the sensor data occurs while the user is engaging in a sleep session, and wherein presenting the at least one updated therapy parameter occurs while the user is engaging in the sleep session.

6. The method of claim 5, wherein the one or more therapy parameters includes an alarm time, wherein the at least one updated therapy parameter includes an updated alarm time, and wherein presenting the at least one updated therapy parameter includes adjusting the alarm time based at least in part on the updated alarm time.

7. The method of claim 1, wherein dynamically generating the at least one updated therapy parameter includes:
receiving target sleep efficiency information;
determining current sleep efficiency information based at least in part on the sensor data;
identifying a difference between the target sleep efficiency information and the current sleep efficiency information; and
generating the updated therapy parameter in response to identifying the difference, wherein the updated therapy parameter is based at least in part on the target sleep efficiency information and the current sleep efficiency information.

8. The method of claim 1, wherein dynamically generating the at least one updated therapy parameter is based at least in part on i) sleep quality information based at least in part on the sensor data, ii) one or more sleep events detected based at least in part on the sensor data, iii) an apnea-hypopnea index calculated based at least in part on the sensor data, iv) sleep stage information based at least in part on the sensor data, or v) any combination of i)-iv).

9-13. (canceled)

14. The method of claim 1, wherein dynamically generating the at least one updated therapy parameters includes:
accessing a historical log associated with the sleep therapy plan; and
generating the at least one updated therapy parameter based at least in part on the historical log.

15-16. (canceled)

17. The method of claim 1, wherein receiving the sensor data from the one or more sensors includes receiving non-contact sensor data from at least one non-contact sensor, and wherein dynamically generating the at least one updated therapy parameter includes:
extracting biomotion information based at least in part on the non-contact sensor data;
identifying body movement information based at least in part on the extracted biomotion information; and
generating the at least one updated therapy parameter based at least in part on the body movement information.

18. The method of claim 1, wherein receiving the sensor data from the one or more sensors includes receiving environment data from i) a temperature sensor; ii) a light sensor; iii) a presence sensor; iv) a microphone; or v) any combination of i-iv; and wherein dynamically generating the at least one updated therapy parameter is based at least in part on the environment data.

19. The method of claim 1, wherein receiving the sensor data from the one or more sensors includes receiving pharmacological data from i) a pharmacological container sensor; ii) a camera; iii) a weight sensor; or iv) any combination of i-iii; and wherein dynamically generating the at least one updated therapy parameter is based at least in part on the pharmacological data.

20. (canceled)

21. The method of claim 1, further comprising:
determining sleep quality information based at least in part on the sensor data, the sleep quality information including i) sleep efficacy information; ii) sleep state information; iii) sleep stage information; iv) detected sleep event information; v) a calculated apnea-hypopnea index; vi) or any combination of i)-v);
generating a sleep therapy plan score based at least in part on the sleep quality information; and
storing the sleep therapy plan score in association with the one or more therapy parameters of the sleep therapy plan.

22-23. (canceled)

24. The method of claim 21, wherein dynamically generating the at least one updated therapy parameter includes:
accessing a historical sleep therapy plan score associated with one or more historical parameters;
comparing the historical sleep therapy plan score with the sleep therapy plan score; and
generating the at least one updated therapy parameter based at least in part on the one or more historical parameters, the one or more parameters, and the comparison between the historical sleep therapy plan score and the sleep therapy plan score.

25. The method of claim 1, wherein receiving the sensor data includes receiving first sensor data while the user is not engaging in a sleep session, and receiving second sensor data while the user is engaging in the sleep session; and wherein dynamically generating the at least one updated therapy parameter is based at least in part on the first sensor data and the second sensor data.

26. A method comprising:
receiving sensor data from one or more sensors, the sensor data being associated with a user;
determining one or more physiological parameters based at least in part on the received sensor data;
generating a sleep disorder prediction based at least in part on the one or more physiological parameters;
identifying a future sleep therapy plan associated with the user;
generating a sleep therapy plan recommendation based at least in part on the generated sleep disorder prediction and the identified sleep therapy plan; and
facilitating application of the sleep therapy plan recommendation to the future sleep therapy plan prior to implementation of the future sleep therapy plan.

27-30. (canceled)

31. The method of claim 26, wherein the sensor data includes subjective user feedback associated with a sleep session, wherein the method further comprises generating a sleep score indicative of a stress level of the user based at least in part on the subjective user feedback, and wherein identifying the future sleep therapy plan is based at least in part on the stress score.

32-33. (canceled)

34. The method of claim 26, further comprising generating a sleep quality log based at least in part on the sensor data, wherein the sleep quality log includes i) sleep state information; ii) sleep stage information; iii) subjective user feedback associated with a sleep session, or iv) any combination of i)-iii); wherein identifying the future sleep therapy plan is based at least in part on the sleep quality log.

35-37. (canceled)

38. The method of claim 26, wherein the sensor data includes non-contact sensor data from one or more non-contact sensors, and wherein determining the one or more physiological parameters includes:
extracting biomotion information based at least in part on the non-contact sensor data; and
determining a body movement parameter based at least in part on the extracted biomotion information, wherein generating the sleep disorder prediction is based at least in part on the body movement parameter.

39. The method of claim 26, identifying the future sleep therapy plan includes:
determining one or more sleep duration parameters associated with a target sleep duration used in the future sleep therapy plan, and wherein the sleep therapy plan recommendation includes i) suggested changes to at least one of the one or more sleep duration parameters, ii) suggested values for at least one of the one or more sleep duration parameters, or iii) both i and ii;
automatically providing one or more default therapy parameters;
determining one or more therapy parameters associated with the futures sleep therapy plan, wherein the one or more sleep therapy parameters includes: i) a sleep restriction parameter; ii) a sleep compression parameter; iii) a pharmacological parameter; iv) a sleep onset latency parameter; v) a sleep environment parameter;
vi) a pre-sleep activity parameter; or vii) any combination of i-vi; or determining that the future sleep therapy plan is a cognitive behavior therapy for insomnia (CBTi) plan.

40-48. (canceled)

49. A system comprising:
a control system including one or more processors; and
a memory having stored thereon machine readable instructions;
wherein the control system is coupled to the memory, and wherein the one or more processors of the control system are configured to execute the machine readable instructions in the memory to:
receive sensor data from one or more sensors, the sensor data being associated with a user engaging in a sleep therapy plan;
receive one or more therapy parameters associated with the sleep therapy plan;
dynamically generate at least one updated therapy parameter associated with the sleep therapy plan based at least in part on the one or more therapy parameters and the received sensor data: and
present the at least one updated therapy parameter in association with the sleep therapy plan.

50-52. (canceled)
